(19) United States
(12) Patent Application Publication
(10) Pub. No.: US 2010/0168665 A1
(43) Pub. Date: Jul. 1, 2010

(54) SEGMENTED BALLOON FOR CATHETER TIP DEFLECTION

(75) Inventor: Gregory J. Skerven, Kernersville, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 12/346,398

(22) Filed: Dec. 30, 2008

(51) Int. Cl. A61M 25/10

(52) U.S. Cl. 604/95.03; 604/98.01; 604/101.01

(57) ABSTRACT

A balloon catheter is provided that may be used to maneuver around tortuous body lumens. The balloon catheter comprises one or more balloons disposed about a tip of the shaft. Each balloon includes a dedicated lumen to allow inflation of each balloon independent of the others. Inflation of a balloon causes the shaft tip to deflect in a direction that is oriented opposite of the inflated balloon. The bent orientation of the shaft tip allows the catheter and a guide wire to be maneuvered through tortuous body lumens.

### BACKGROUND

The invention generally relates to a medical device which selectively directs a shaft tip and/or wire guide into a branched body passageway.

Navigating a medical device through a body passage can be difficult when attempting to maneuver within a selected branching pathway, such as a bifurcated duct or vessel. For example, most wire guides lack the ability to maneuver in a particular direction, especially when the direction is against the natural pathway that the wire guide prefers to take.

An example of an area of the body where this poses a problem is the biliary tree, where wire guides are often introduced prior to procedures such as endoscopic retrograde cholangiopancreatography (ERCP), which is a diagnostic visualization technique commonly used with a sphincterotome. The biliary tree includes bifurcations at the junction of the biliary and pancreatic ducts, and between the right and left hepatic ducts. The anatomy of the biliary tree can make navigation of the wire guide into the desired branch of the bifurcation difficult.

Current devices used to direct wire guides have wires attached to the tips of the devices which are tensioned to create a desired tip orientation. Other devices are designed with ramps to deflect the wire guide out of a side port of the device. Both designs suffer drawbacks such as requiring large lumens and offering resistance to wire movement when the device is in a tortuous configuration, such as a branched body passageway.

In view of the difficulties of successfully navigating into and within a branched body passageway, there is a need for a medical device that can reliably gain access to and navigate through a branched body passageway.

### SUMMARY

The invention may include any of the following aspects in various combinations and may also include any other aspect described below in the written description or in the attached drawings.

In a first aspect, a balloon catheter for use in a body lumen is provided. The balloon catheter comprises a shaft having a distal end and a proximal end. The shaft has one or more inflation lumens in which the one or more inflation lumens proximally extends and terminates into one or more corresponding inflation lumen ports. Each of the corresponding one or more inflation lumen ports is configured to be in fluid communication with a pressurizable inflation source. One or more balloons is disposed circumferentially about the distal end of the shaft. Each of the one or more balloons is disposed along a corresponding portion of a circumference of the distal end of the shaft. Each of the one or more balloons has a separate interior chamber corresponding with the one or more inflation lumens. The one or more balloons has a structure configured for expansion of the interior chamber, such that expansion of the one or more balloons creates an asymmetrical force sufficient to bend the distal end of the shaft in a lateral direction.

In a second aspect, a balloon catheter for use in a body lumen is provided. The balloon catheter comprises a shaft having a distal end and a proximal end. The shaft has a first inflation lumen. The first inflation lumen proximally extends and terminates into a corresponding inflation lumen port. The inflation lumen port is configured to be in fluid communication with a pressurizable inflation source. A first balloon spans a first arc region circumferentially about an outer surface of the distal end of the shaft. The first balloon has a first interior chamber in fluid communication with the first inflation lumen. The first balloon is configured to expand from a deflated state to an expanded state. The expansion creates a first force sufficient to bend the distal end of the shaft in a first lateral direction to produce a first deflection at the distal end along a direction of the first force.

In a third aspect, a method of advancing a device through a tortuous body lumen is provided. A balloon catheter is provided comprising a shaft having a distal end and a proximal end. The shaft has a first inflation lumen. The first inflation lumen proximally extends and terminates into a corresponding first inflation lumen port. The first inflation lumen port is configured to be in fluid communication with a pressurizable inflation source. A first balloon spans a first arc region circumferentially about an outer surface of the distal end of the shaft. The first balloon has a first interior chamber in fluid communication with the first inflation lumen. Inflation fluid is injected through the port with the inflation source to inflate the first balloon. The distal end of the shaft is asymmetrically loaded with a first force. The distal end of the shaft is bent along the direction of the first force in a first direction to create a first bent orientation. Having bent the distal end, the distal end of the shaft is advanced along the first direction to gain access through the body lumen.

### BRIEF DESCRIPTION OF SEVERAL VIEWS OF THE DRAWINGS

The invention may be more fully understood by reading the following description in conjunction with the drawings, in which:

FIG. 1 is a side view of a balloon catheter including four inflation ports in fluid communication with their respective balloons;

FIG. 2 shows an end cross-sectional view of the balloon catheter of FIG. 1;

FIG. 3 shows a lateral cross-sectional view of the balloon catheter of FIGS. 1 and 2;

FIG. 4 shows a method of using the balloon catheter in which selective inflation of the balloons causes the distal end of the balloon catheter to deflect into the biliary duct;

FIG. 5 shows a method of using the balloon catheter in which selective inflation of the balloons causes the distal end of the balloon catheter to deflect into the pancreatic duct;

FIG. 6 is an embodiment of a deflated balloon fabricated in a pre-curved shape;

FIG. 7 is the balloon of FIG. 6 inflated to its pre-curved state;

FIG. 8 is an another embodiment of a deflated balloon attached to a catheter shaft at discrete locations; and

FIG. 9 is the balloon of FIG. 8 inflated to an expanded state; and

FIG. 10 shows an outer reinforcement member helically wrapped around a distal end of catheter shaft to create a greater durometer relative to the nonreinforced portion of shaft.

### DETAILED DESCRIPTION

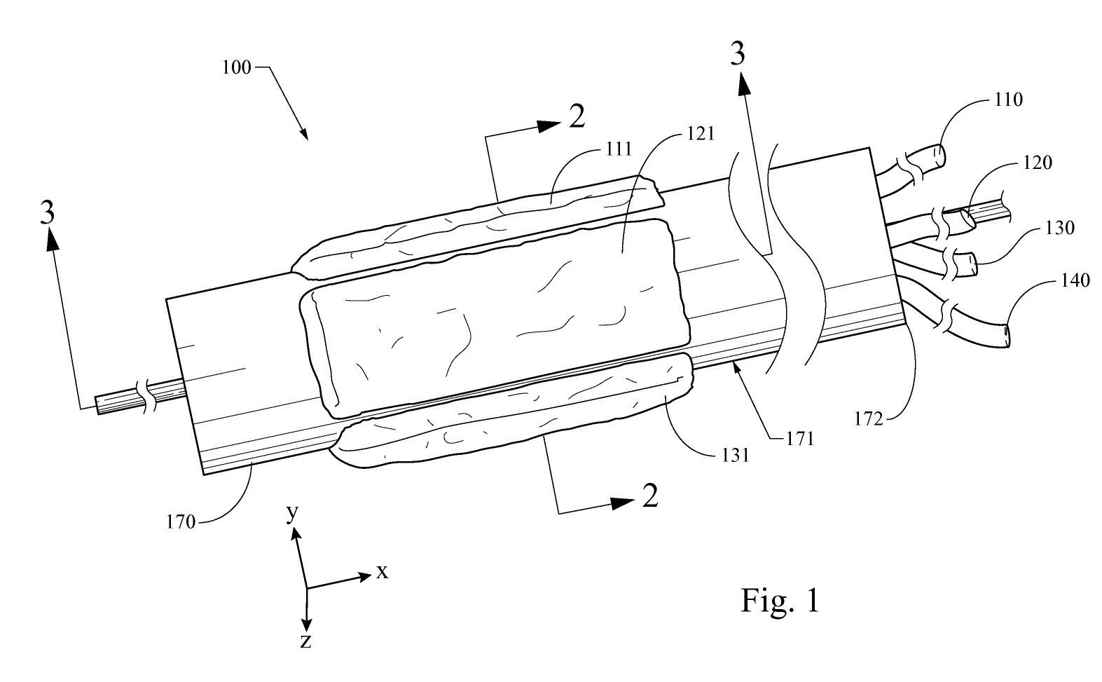

FIG. 1 is a lateral view of a balloon catheter 100 including balloons 111, 121, 131, and 141 disposed about a distal end 170 of catheter shaft 171. Balloon 141 is not shown in FIG. 1 because the balloon 141 is located into the plane of the page. Each of the balloons 111, 121, 131, and 141 extends about a corresponding portion of the circumference of the outer surface of the distal end 170 of the shaft 171. Generally speaking, expansion of each of the balloons 111, 121, 131, and 141 is independently controlled with respect to the other balloons 111, 121, 131, and 141. The ability to selectively inflate each of the balloons 111, 121, 131, and 141 enables the distal end 171 of the shaft 170 to deflect in a controlled manner. In particular, inflation of one of the balloons 111, 121, 131, and 141 causes the distal end 171 to deflect in a direction that is oppositely disposed of the inflated balloon 111, 121, 131, and 141. The bent orientation of the shaft 171 allows the catheter 100 to be maneuvered through tortuous body lumens.

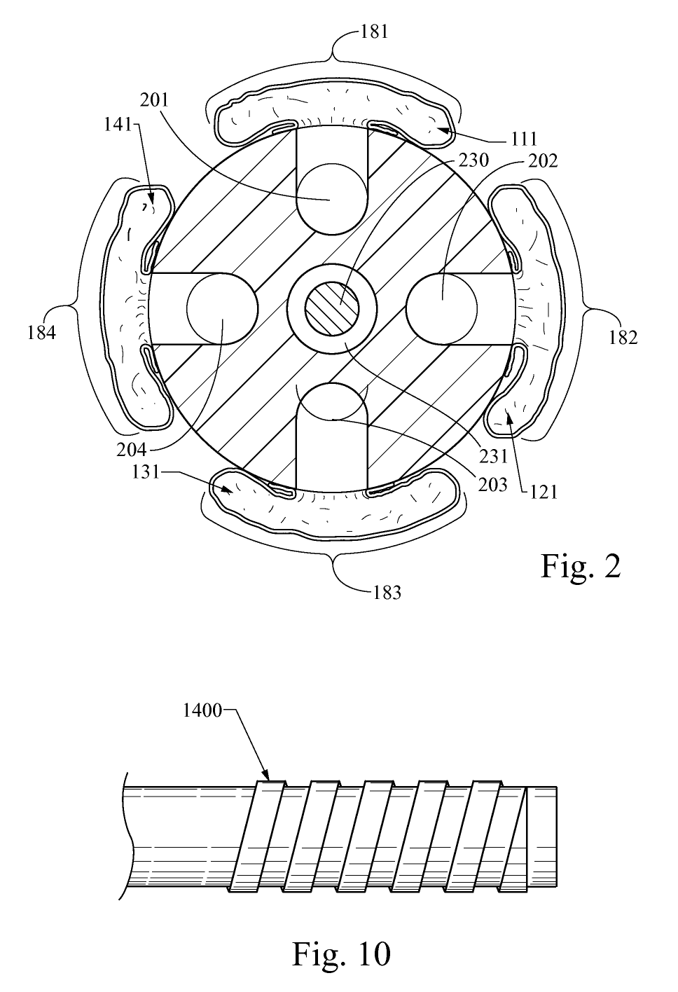

The balloons 111, 121, 131, and 141 may span any circumferential length. In the embodiments shown in FIGS. 1 and 2, each of the balloons 111, 121, 131, and 141 spans an arc of about 90° about the outer surface of the shaft 171. Referring to FIG. 2, balloon 111 is disposed along arc region 181. Balloon 121 is disposed along arc region 182. Balloon 131 is disposed along arc region 183, and balloon 141 is disposed along arc region 184. Balloon 111 is oriented about 90 degrees from balloons 121 and 141 and about 180 degrees from balloon 131.

Each of the balloons 111, 121, 131, 141 includes a dedicated inflation lumen 201, 202, 203, and 204 to allow selective expansion of each of the balloons 111, 121, 131, and 141. The interior regions of the balloons 111, 121, 131, and 141 are in fluid communication with corresponding inflation lumens 201, 202, 203, and 204. Inflation fluid may be introduced through one or more of the inflation ports 110, 120, 130, 140 (FIG. 1). As an example, introducing inflation fluid through port 110 causes balloon 111 to inflate and expand. Inflation fluid may include any type of fluid or gas known in the art, including but not limited to saline and air. Balloon 111 may inflate to a desired expanded state while the other balloons 121, 131, and 141 remain deflated about the outer surface of the shaft 171. The inflation of balloon 111 creates a force against the distal end 171 resulting in asymmetrical loading of the shaft tip. The asymmetrical loading causes the shaft 171 to deflect. In use, a combination of inflated and deflated balloons 111, 121, 131, and 141 is created to deflect the distal end 171 of the shaft tip in a direction that allows maneuverability through tortuous body lumens.

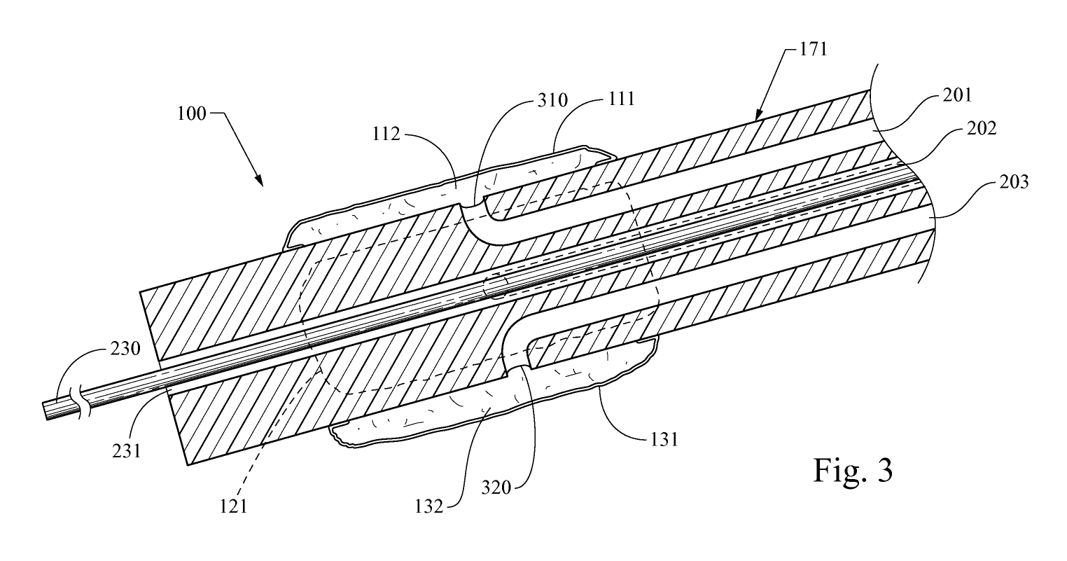

FIG. 3 shows a cross-sectional view of the balloon catheter 100. Inflation lumen 201 feeds into the interior region 112 of balloon 111 through an aperture 310. The aperture 310 is an opening which extends along a radial direction from the inflation lumen 201 to the interior region 112 of the balloon 111. The aperture 310 provides a pathway through which the inflation fluid enters into the interior region 112 of the balloon 111 to inflate balloon 111 to an expanded state.

FIG. 3 also shows inflation lumen 203 feeding into the interior region 132 of balloon 131 through aperture 320. The aperture 320 provides an opening from the inflation lumen 203 into the interior region 132 of segmented balloon 131 through which inflation fluid enters to inflate segmented balloon 131 to an expanded state. Segmented balloon 131 is oppositely disposed from balloon 111. Balloon 121 and its respective inflation lumen 202 are shown in phantom lines in FIG. 3.

The balloon catheter 100 may include a wire guide 230 extending through a wire guide lumen 231, as shown in the Figures. FIG. 2 shows that the wire guide lumen 231 is situated about the center of the shaft. The inflation lumens 201, 202, 203, and 204 are situated radially outward from the wire guide lumen 231.

Still referring to FIG. 2, each of the balloons 111, 121, 131, and 141 is shown in its deflated state about the outer surface of the shaft 171. Each of the balloons 111, 121, 131, and 141 is preferably circumferentially spaced apart a predetermined amount so that inflation of adjacently disposed balloons 111, 121, 131, and 141 can be achieved without significant interference of the balloons 111, 121, 131, and 141.

The degree of bending upon inflation of a single balloon is dependent upon how much inflation fluid is injected into the interior of each of the balloons 111, 121, 131, and 141, as well as the dimensions and the volume capacity of each of the balloons 111, 121, 131, and 141.

Although the embodiments have been described with a balloon catheter 100 having four balloons 111, 121, 131, and 141, more than four or less than four balloons are contemplated. The exact number of balloons may be dependent upon the size of the particular body lumen that the balloon catheter is being navigated and maneuvered within.

Other configurations of the balloons 111, 121, 131, and 141 are contemplated. As one example, the balloons 111, 121, 131, and 141 may be longitudinally staggered along the distal end 171 to create a spiral arrangement. Such a configuration may prevent the distal end of the shaft 171 to be deflected into a spiral orientation.

Additionally, different sized balloons can be placed about the shaft based on the particular application. For example, if relatively greater deflection of the catheter tip is desired to be created to navigate through a tortuous body lumen, then a larger sized balloon may be placed along one of the arc regions 181, 182, 183, and 184 of the shaft 171 outer surface. This may be achieved by either increasing the length and/or diameter of the balloon.

The deflection characteristics of the shaft 171 can also be altered by modifying the manner, location, and size of the attachment between the balloon and the shaft 171 as will now be explained. FIGS. 6 and 7 show one example of a balloon 601 that induces a curve along the shaft 171 when the balloon 601 inflates from a deflated state (FIG. 6) to an inflated state (FIG. 7). During manufacturing, the balloon 601 may be extruded or molded into a curve shape. The balloon 601 is thereafter attached to the distal end 170 of the outer surface of the shaft 171 as known in the art. FIG. 6 shows the balloon 601 in a deflated state along the shaft 171. When the balloon 601 inflates to an expanded state, it reverts to its curved shape (e.g., banana shape). The curved shape of the balloon 601 transmits a force along the outer surface of the shaft 171 to induce a curve along the distal end 170 of shaft 171, thereby causing the shaft 171 to bend as shown in FIG. 7. The balloon 601 is preferably formed from a noncompliant material as known in the art and the shaft 171 is preferably formed from a pliable material as known in the art such that transmission of the force from the curved balloon 601 to the shaft 171 causes the shaft 171 to laterally deflect and bend.

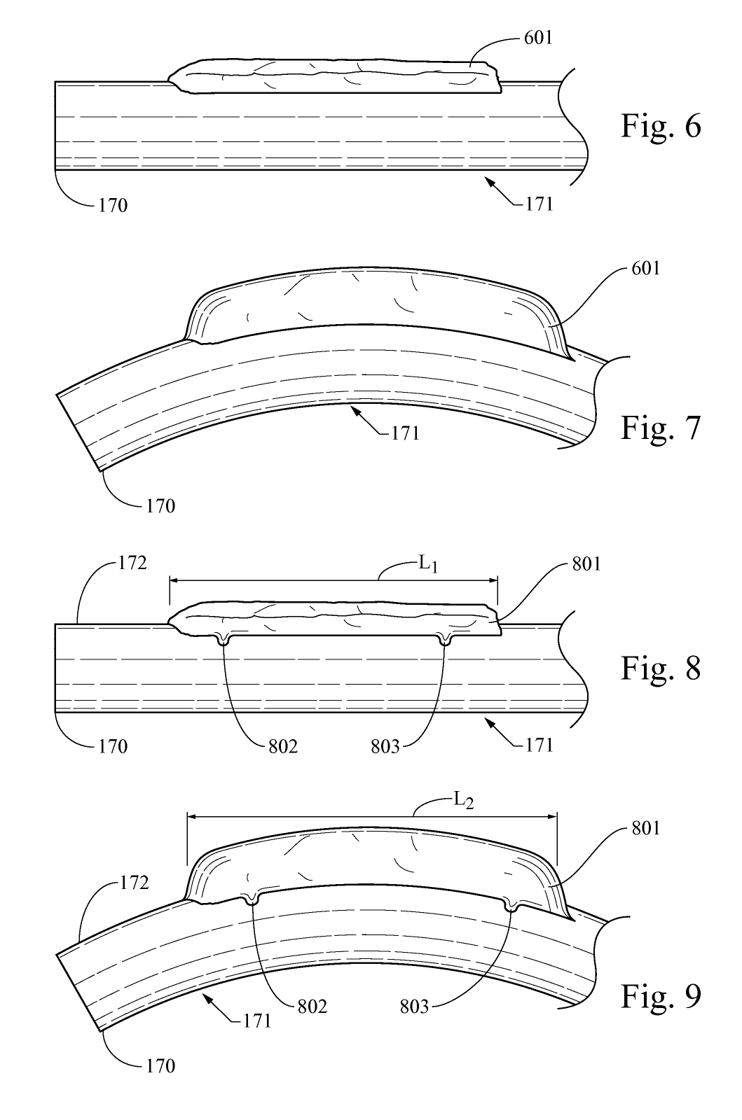

FIGS. 8 and 9 show another manner of attaching the balloon 801 to the shaft 171. FIG. 8 shows a balloon 801 in a substantially linear configuration when deflated and having a longitudinal length of L₁. The balloon 801 is attached at discrete locations 802 and 803 to shaft 171 along the outer surface 172 of shaft 171. The locations 802 and 803 have a spaced apart distance slightly less than the overall longitudinal length of the inflated balloon 801. The balloon 801 may be attached along any number of discrete locations so long as the spacing of attachments at the ends of the balloon 801 is less than the inflated length of the balloon 801. FIG. 9 shows that as the balloon 801 inflates, the overall longitudinal length of the balloon 801 increases from L₁ to L₂. Such an increase in longitudinal length of the balloon 801 tends to elongate or stretch the outer surface 172 of the shaft 171, thereby inducing a curve along the distal end 170 of the shaft 171. The balloon 601 is preferably formed from a noncompliant material as known in the art and the shaft 171 is preferably formed from a pliable material as known in the art such that transmission of a force from the curved balloon 601 to the shaft 171 causes the shaft 171 to elongate or stretch and bend in a lateral direction. Although FIGS. 8 and 9 show two locations 802 and 802 at which the balloon 801 is attached to outer surface 172 of shaft 171, more than two locations are contemplated so long as the end-most attachments have a spaced apart distance slightly less than the overall longitudinal length of the inflated balloon 801. For example, referring to FIG. 8, the deflated balloon 801 may be connected to the shaft 171 at about the midpoint of the longitudinal length of the deflated balloon 801.

The embodiments of FIGS. 6-9 may occur inside or outside a body lumen. Other means for causing an inflated balloon to induce a curve to shaft 171 is contemplated. Generally speaking, any means for causing the balloon to undergo a change in its dimensional shape which is transmitted to the distal end 170 of the shaft 171 is contemplated.

In addition and separate from the above embodiments described in FIGS. 6-9, one or more balloons 111, 121, 131, and 141 can be inflated and expanded to engage a surface of a body lumen so as to push the distal end 170 of the shaft 171 away from the surface of the body lumen. The one or more balloons 111, 121, 131, and 141 may be formed from either a noncompliant or compliant material.

The shaft 171 may be formed from any biocompatible material. Preferably, the shaft 171 is formed from a compliant material as known in the art that readily undergoes bending when incurring a load. Suitable compliant materials include polyurethane, silicone, latex, polyethylene or polyolefin copolymers. The balloons 111, 121, 131, 141 may be formed from compliant or noncompliant material as known to one of ordinary skill in the art. However, as described with respect to the embodiments of FIGS. 6-9, noncompliant materials are preferred to facilitate the transfer of forces from the balloon to the shaft 171.

The shaft 171 may be made by any methods known to one of ordinary skill in the art, including but not limited to extrusion, pultrusion, injection molding, transfer molding, flow encapsulation, fiber winding on a mandrel, or lay-up with vacuum bagging. A variety of suitable materials may be used, so long as the materials provide desired flexibility of the shaft 171. For example, suitable materials include surgical stainless steel or biologically compatible metals, polymers, plastics, alloys (including super-elastic alloys), or composite materials that are either biocompatible or capable of being made biocompatible. Other suitable materials (natural, synthetic, plastic, rubber, metal, or combination thereof) are preferably strong yet flexible and resilient comprising, for by way of illustration and not by way of limitations, elastomeric materials such as and including any latex, silicone, urethane, thermoplastic elastomer, nickel titanium alloy, polyether ether-ketone (“PEEK”), polyimide, polyurethane, cellulose acetate, cellulose nitrate, silicone, polyethylene terephthalate (“PET”), polyamide, polyester, polyorthoester, polyanhydride, polyether sulfone, polycarbonate, polypropylene, high molecular weight polyethylene, polytetrafluoroethylene (“PTFE”), or mixtures or copolymers thereof, polylactic acid, polyglycolic acid or copolymers thereof, polycaprolactone, polyhydroxyalkanoate, polyhydroxy-butyrate valerate, polyhydroxy-butyrate valerate, or another polymer or suitable material.

In one embodiment, the shaft 171 or at least a distal portion therealong may comprise an optional anisotropic material that is, or can be made to be, relatively compliant in an axial direction as compared to a transverse direction. This characteristic is known generally as “anisotropy” (in contrast to “isotropy” where the material characteristics are uniformly independent of direction or orientation within the material). In one embodiment of the invention that uses optional anisotropic material, the specific anisotropic behavior would be achieved by circumferentially reinforcing the shaft 171 so that its “hoop” stiffness (e.g., circumferential stiffness) is higher than its axial stiffness. This could be accomplished by a variety of methods, one of which would be to wrap or wind reinforcing fibers around the shaft 171, or to embed them circumferentially within the material. Consequently, selective inflation of one or more of the balloons 111, 121, 131, and 141 would generate forces within the material of shaft 171 that result in a desired deflection force.

In the axial direction, the specific type of elastic behavior used in formation of shaft 171 may have an impact on the extent to which deflection of shaft 171 along its distal end 170 is created. For example, if an elastomeric material is used (e.g., rubber), which by definition has a distensibility in the range of 200%-800%, then inflation of one or more of the balloons 111, 121, 131, and 141 may generate forces sufficient to generate a relatively large angular deflection, resulting in a sharp (short radius) turn. If a substantially non-elastomeric material is used (e.g., conventional catheter materials) then relatively smaller angular deflections will be created, resulting in a less sharp turn (i.e., large-radius bend). Accordingly, selection of a suitable material may depend, at least in part, on the degree of bending desired when navigating balloon catheter 100 within a particular branched body passageway.

The distal end 170 of shaft 171 may comprise a greater durometer (i.e., harder, more stiff) relative to the proximal portion of shaft 171 so as to enable the distal end 170 to bend but resist kinking during deflection of shaft 171 therealong. Means for achieving the greater durometer include, but are not limited to, affixing an internal or outer reinforcement member to distal end 170, such as a spring, coil, mesh, wire, fiber, or cannula. FIG. 10 shows an outer reinforcement member 1400 helically wrapped or compression fitted around a distal end 170 of shaft 171 to create a distal region having a greater durometer compared to nonreinforced portions of shaft 171. The reinforcement member 1400 may be formed from any medical grade metals and alloys or other biocompatible materials which provide sufficient structural reinforcement.

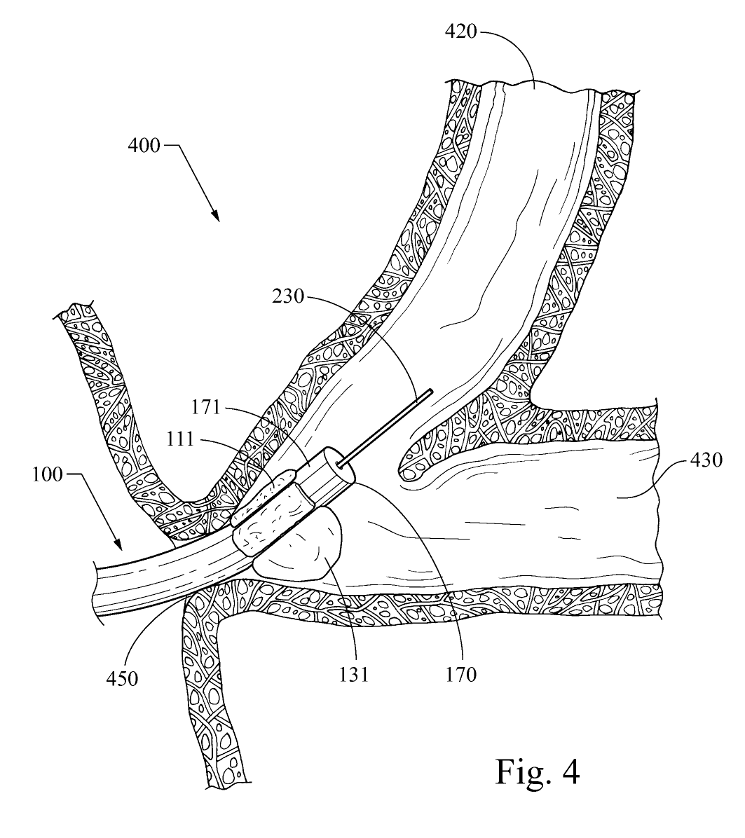

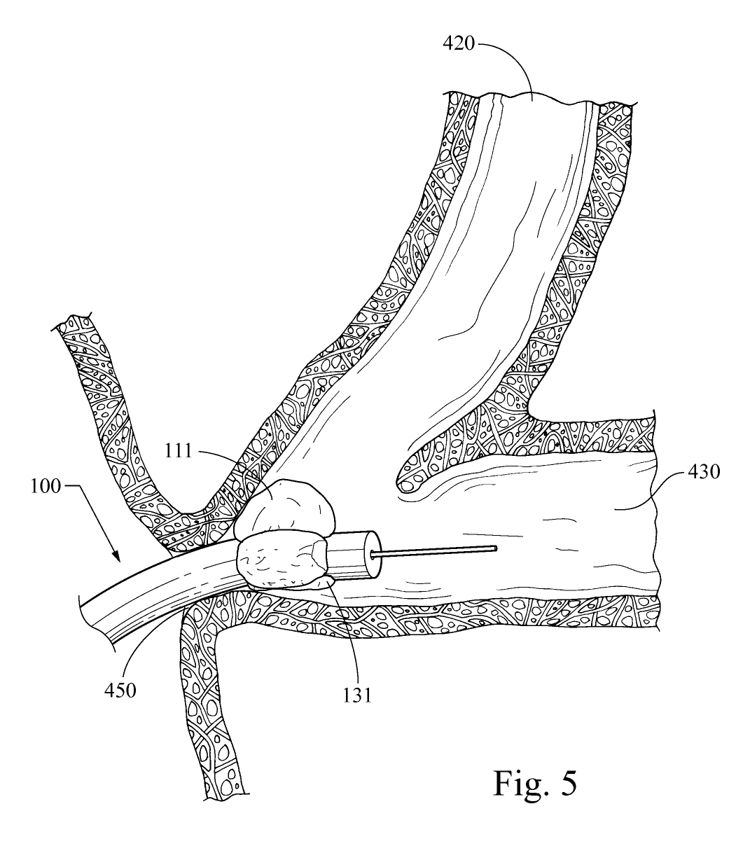

A method for maneuvering a balloon catheter 100 within a selected branch of a body passageway will now be described in conjunction with FIGS. 4 and 5. FIG. 4 shows a biliary tree 400, which is a common branched body passageway that can be difficult to navigate within. Selective inflation of the balloons 111, 121, 131, 141 can overcome the navigation and maneuverability difficulties. The catheter 100 is advanced over a wire guide 230 through the esophagus, gastrointestinal lumen, and into the duodenum until it is either positioned in close proximity to the papilla 450 or is advanced through the papilla 450 as shown in FIG. 4. During advancement, all of the balloons 111, 121, 131, and 141 are preferably in their deflated states to create a reduced lateral profile as shown in FIGS. 1 and 2.

After the balloon catheter 100 has been advanced through the papilla 450, inflation fluid is introduced into inflation port 120. The fluid may be introduced from any pressurized fluid source. The inflation fluid flows into port 120 (FIG. 1) and thereafter flows within inflation lumen 202 (FIG. 3). The fluid travels through the lumen 202 and enters the interior region 132 of balloon 131 through its corresponding aperture 320 (FIG. 3). Inflation fluid continues to be introduced into interior region 132 until balloon 131 inflates to a predetermined expanded state that is sufficient to exert a deflecting force on shaft 171, as shown in FIG. 4. The deflecting force deflects the distal end 170 of shaft 171 into a bent configuration in which the distal end 170 of the shaft 171 is oriented towards the biliary duct 420. With the balloon 131 still expanded and the distal end 170 in the desired bent configuration, the wire guide 170 (FIG. 1) is advanced distally beyond the distal end of the catheter shaft 171 and into the biliary duct 420, as shown in FIG. 4. After the wire guide 230 has sufficiently traveled into the biliary duct 420, the balloon 131 may be deflated and the catheter 100 may be advanced over wire guide 230 and into biliary duct 420. As advancement of the distal end 170 continues, the wire guide 230 may be further distally advanced so that the distal end of the wire guide 230 distally travels further into the biliary duct 420. Alternatively, the catheter 100 may be withdrawn so that the wire guide 230 may used to advance other elongate medical devices therealong.

FIG. 5 shows the balloon catheter 100 being navigated into pancreatic duct 430. After the balloon catheter 100 has been advanced through the papilla 450, inflation fluid is introduced into inflation port 110. The inflation fluid flows into port 110 and thereafter travels through inflation lumen and thereafter enters the interior region 112 of balloon 111 through aperture 310 (FIG. 3). Inflation fluid continues to be introduced into interior region 112 through lumen 201 until balloon 111 inflates to a predetermined expanded state that is sufficient to exert a deflecting force on the distal end 170 of shaft 171, as shown in FIG. 5. The deflecting force deflects the distal end 170 of the shaft 171 into a bent configuration oriented in about a 3 o'clock position so that the distal end 170 of the shaft 171 is oriented towards the pancreatic duct 420.

With balloon 111 remaining in the expanded configuration and the distal end 170 in the desired bent configuration, the wire guide 230 is advanced distally beyond the distal end 170 (FIG. 1) of the catheter shaft 171 and into the pancreatic duct 420 as shown in FIG. 5. After the wire guide 230 has been sufficiently advanced into the pancreatic duct 420, the balloon 111 may be deflated and the distal end 170 of the shaft 171 advanced therealong. As advancement of the distal end 170 into pancreatic duct 420 continues, the wire guide 230 may be further distally advanced so that the distal end of the wire guide 230 distally travels further into the pancreatic duct 420.

Although the above method has been described using a conventional wire guide as known in the art, the balloon catheter 100 of the present invention may also be used to direct other elongate member members. For example, an elongate fiber having light propagating properties, along its length, such as an optical fiber, may extend through the wire guide lumen 231 of the balloon catheter 100 so as to selectively advance the distal end of the elongate fiber through the biliary duct 430 or pancreatic duct 420. Transmission of light along the optical fiber may further enable the physician to view its advancement into the desired duct 420 or 430.

It should be understood that selected navigation and maneuverability of the catheter 100 within the biliary duct 420 or pancreatic duct 430 are merely exemplary methods and that the balloon catheter 100 may be used to maneuver within other branched body passageways.

The above methods as explained in conjunction with FIGS. 4 and 5 illustrate selective inflation of a single balloon to deflect the catheter tip and maneuver within a branched body lumen. Alternatively, more than a single balloon may be inflated in suitable applications. For example, there may be instances where the shaft 171 tip needs to be deflected in other or multiple planes to maneuver within a tortuous body lumen. Accordingly, two adjacently disposed balloons of balloon catheter 100 may be inflated. For example, referring to FIG. 1, inflation of balloon 111 and inflation of balloon 121 may cause the distal end 170 of shaft 171 to bend in the x-z and y-x planes (FIG. 1).

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. A balloon catheter for use in a body lumen, the balloon catheter comprising:
a shaft having a distal end and a proximal end, the shaft having one or more inflation lumens, the one or more inflation lumens proximally extending and terminating into one or more corresponding inflation lumen ports, each of the corresponding one or more inflation lumen ports configured to be in fluid communication with a pressurizable inflation source;
one or more balloons disposed circumferentially about the distal end of the shaft, each of the one or more balloons disposed along a corresponding portion of a circumference of the distal end of the shaft, each of the one or more balloons having a separate interior chamber corresponding with the one or more inflation lumens, wherein the one or more balloons has a structure configured for expansion of the interior chamber, and further wherein expansion of the one or more balloons creates an asymmetrical force sufficient to bend the distal end of the shaft in a lateral direction.

2. The balloon catheter of claim 1, further comprising one or more apertures, each of the one of more apertures extending along a radial direction from the one or more inflation lumens to the separate interior chamber of each of the one or more balloons.

3. The balloon catheter of claim 1, wherein each of the corresponding portions of the circumference spans an arc of about 90 degrees.

4. The balloon catheter of claim 1, the pressurizable inflation source further comprising a pumping device configured for pumping inflation fluid into the one or more inflation lumens to expand the one or more balloons.

5. The balloon catheter of claim 1, wherein the shaft is formed from a compliant material and the one or more balloons are formed from a noncompliant material.

6. The balloon catheter of claim 1, further comprising four balloons, each of the four balloons having substantially equal volume capacity.

7. The balloon catheter of claim 1, further comprising a first, a second, a third, and a fourth balloon, each of the first, the second, the third, and the fourth balloons occupying a corresponding first, a second, a third, and a fourth arc region, the first arc region disposed adjacent to the second and the third arc regions, and the first arc region disposed opposite to the fourth arc region.

8. The balloon catheter of claim 7, wherein inflation of the first balloon in combination with the third or the second balloons causes the distal end of the shaft to deflect along a first plane and a second plane so as to orient into a new bent orientation.

9. The balloon catheter of claim 7, wherein inflation of one of the first, the second, the third, and the fourth balloons causes the distal end of the shaft to deflect along a first plane into a new bent orientation.

10. The balloon catheter of claim 1, wherein the one or more balloons is configured into a spiral arrangement along a longitudinal axis of the shaft.

11. The balloon catheter of claim 1, wherein fewer than each of the one or more balloons is disposed along the corresponding portion of the circumference spanning about 90 degrees.

12. The balloon catheter of claim 1, wherein the bending comprises engaging the one or more expanded balloons against the body lumen so as to push the distal end off from the body lumen.

13. The balloon catheter of claim 1, wherein the one or more balloons comprises a curved shape when expanded.

14. The balloon catheter of claim 1, wherein the one or more balloons is attached at a first discrete location and a second discrete location, and further wherein a longitudinal distance between the first and the second discrete locations is less than a longitudinal length of the one or more inflated balloons.

15. A balloon catheter for use in a body lumen, the balloon catheter comprising:
a shaft having a distal end and a proximal end, the shaft having a first inflation lumen, the first inflation lumen proximally extending and terminating into a corresponding inflation lumen port, the inflation lumen port configured to be in fluid communication with a pressurizable inflation source; and
a first balloon spanning a first arc region circumferentially about an outer surface of the distal end of the shaft, the first balloon having a first interior chamber in fluid communication with the first inflation lumen, the first balloon configured to expand from a deflated state, wherein the expansion creates a first force sufficient to bend the distal end of the shaft in a first lateral direction to produce a first deflection at the distal end along a direction of the first force.

16. The balloon catheter of claim 15, further comprising a second balloon, the second balloon spanning a second arc region circumferentially about an outer surface of the distal end of the shaft, the second balloon having a second interior chamber in fluid communication with a second inflation lumen, wherein the second arc region is adjacently disposed to the first arc region.

17. The balloon catheter of claim 16, wherein inflation of both the first and the second balloons are configured to produce a second defection greater than the first deflection.

18. A method of advancing a device through a tortuous body lumen, comprising the steps of:
(a) providing a balloon catheter comprising:
a shaft having a distal end and a proximal end, the shaft having a first inflation lumen, the first inflation lumen proximally extending and terminating into a corresponding first inflation lumen port, the first inflation lumen port configured to be in fluid communication with a pressurizable inflation source;
a first balloon spanning a first arc region circumferentially about an outer surface of the distal end of the shaft, the first balloon having a first interior chamber in fluid communication with the first inflation lumen;
(b) injecting inflation fluid through the port with the inflation source;
(c) inflating the first balloon;
(d) asymmetrically loading the distal end of the shaft with a first force;
(e) bending the distal end of the shaft along the direction of the first force in a first direction to create a first bent orientation; and
(f) advancing the distal end along the first direction to gain access through the body lumen.

19. The method of claim 18, further comprising the step of providing a second balloon disposed at a second arc region adjacent to the first arc region.

20. The method of claim 19, further comprising the step of inflating the second balloon to bend the distal end along a first plane and a second plane into a new second bent orientation to gain access through the body lumen.

21. The method of claim 18, wherein step (e) further comprises engaging the first balloon against the body lumen so as to push the distal end off from the body lumen and deflect the distal end of the shaft.

22. The method of claim 18, wherein step (e) further comprises inflating the balloon to a pre-curved expanded state, the pre-curved expanded state transmitting a force along the shaft to induce a curve along the distal end of the shaft and deflect the distal end of the shaft.

23. The method of claim 18, wherein step (e) further comprises inflating the balloon to stretch the outer surface of the shaft and induce a curve along the distal end of the shaft.

24. The method of claim 18, further comprising the step of advancing a wire guide through a wire guide lumen of the balloon catheter, wherein a distal portion of the wire guide is advanced through the wire guide lumen so as to extend beyond the bent distal end of the shaft and be directed into a target branch of the body lumen.
